(19) United States
(12) Patent Application Publication
(10) Pub. No.: US 2023/0274821 A1
(43) Pub. Date: Aug. 31, 2023

(54) SYSTEMS AND METHODS TO PROVIDE REAL-TIME FEEDBACK FOR PATIENT WAIT TIME

(71) Applicant: KONINKLIJKE PHILIPS N.V., EINDHOVEN (NL)

(72) Inventors: Lucas OLIVEIRA, WILMINGTON, MA (US); Jin LIU, MALDEN, MA (US)

(21) Appl. No.: 18/015,988

(22) PCT Filed: Jul. 9, 2021
(86) PCT No.: PCT/EP2021/069099

§ 371 Date: Jan. 13, 2023

Related U.S. Application Data
(60) Provisional application No. 63/052,486, filed on Jul. 16, 2020.

(51) Int. Cl. G16H 40/20; G16H 10/60; G16H 50/70

(52) U.S. Cl. CPC G16H 40/20; G16H 10/60; G16H 50/70

(57) ABSTRACT

A non-transitory computer readable medium (26) stores instructions executable by at least one electronic processor (20) to perform a patient wait time tracking method (100). The method includes: extracting clinical and operational features for respective patients of a queue of patients scheduled for examination; estimating patient-specific examination time durations (39) for medical examinations or procedures of the respective patients of the queue based on the extracted clinical and operational features of the respective patients of the queue; estimating a wait time (41) for a query patient of the patients of the queue based on the estimated examination time durations of patients ahead of the query patient in the queue; and transmitting the estimated wait time to a user interface (44) for presentation.

### FIELD

The following relates generally to the medical arts, patient appointment tracking arts, patient wait time monitoring arts, mobile application arts, and related arts.

### BACKGROUND

Patient satisfaction for healthcare providers has been playing an increasingly important role under the transition to value-based healthcare. One of the key factors that affect patient satisfaction is a long and unpredictable waiting time for a scheduled examination or procedure. Research shows that patients use average appointment wait times as one deciding factor when choosing a new clinician. Patients would be less frustrated if they knew how long the wait time would be, and an estimate of how long the wait time will be can help alleviated frustration.

Patient satisfaction affects hospital revenue not only through value-based healthcare payment models but also through patients’ visiting preference. Regarding the latter, patients may consider online reviews written by past patients in selecting healthcare providers; and poor online reviews are often a consequence of past patients experiencing long or unexpected delays. Furthermore, patients who expect/experience long and unpredictable waiting time may end up not visiting hospitals or leaving hospital without being seen, which might cause long-term health problems for patients as well as more complex health conditions with increased healthcare costs.

The following discloses new and improved systems and methods to overcome these problems and others.

### SUMMARY

In one disclosed aspect, a non-transitory computer readable medium stores instructions executable by at least one electronic processor to perform a patient wait time tracking method. The method includes: extracting clinical and operational features for respective patients of a queue of patients scheduled for examination; estimating patient-specific examination time durations for medical examinations or procedures of the respective patients of the queue based on the extracted clinical and operational features of the respective patients of the queue; estimating a wait time for a query patient of the patients of the queue based on the estimated examination time durations of patients ahead of the query patient in the queue; and transmitting the estimated wait time to a user interface for presentation.

In another aspect, an apparatus includes: a database storing patient data related to patients of a queue of patients scheduled for examination, and a display device upon which a user interface is implemented. At least one electronic processor is programmed to: extract patient features for the respective patients of the queue of patients from the database; estimate patient-specific examination time durations for medical examinations of the respective patients of the queue based on the extracted patient features of the respective patients of the queue; estimate a wait time for a query patient of the patients of the queue based on the estimated examination time durations of patients ahead of the query patient in the queue; and transmit the estimated wait time to the user interface for presentation.

In another aspect, a patient wait time tracking method includes: extracting clinical and operational features for respective patients of a queue of patients scheduled for examination; estimating patient-specific examination time durations for medical examinations of the respective patients of the queue based on the extracted clinical and operational features of the respective patients of the queue; applying a workflow model for the medical examinations of the respective patients of the queue; estimating a wait time for a query patient of the patients of the queue based on the estimated examination time durations of patients ahead of the query patient in the queue and the workflow model; and transmitting the estimated wait time to a user interface for presentation.

One advantage resides in providing an estimated wait time for a patient for a medical procedure.

Another advantage resides in increased patient satisfaction.

Another advantage resides in allowing patients to not have to wait in a waiting room of a medical facility for a medical procedure.

Another advantage resides in providing real-time updates to a patient regarding a wait time for a medical procedure.

Another advantage resides in using historical patient data and medical procedure workflow data to estimate and update a wait time for a patient.

Another advantage resides in increasing an operational performance of a medical facility by providing estimated wait times to patients.

A given embodiment may provide none, one, two, more, or all of the foregoing advantages, and/or may provide other advantages as will become apparent to one of ordinary skill in the art upon reading and understanding the present disclosure.

### BRIEF DESCRIPTION OF THE DRAWINGS

The disclosure may take form in various components and arrangements of components, and in various steps and arrangements of steps. The drawings are only for purposes of illustrating the preferred embodiments and are not to be construed as limiting the disclosure.

FIG. 1 diagrammatically shows an illustrative apparatus for providing remote assistance in accordance with the present disclosure.

FIG. 2 shows an example of an example user interface generated by the apparatus of FIG. 1.

FIG. 3 shows an example flow chart of operations suitably performed by the apparatus of FIG. 1.

### DETAILED DESCRIPTION

Patient satisfaction is adversely affected by unanticipated wait times for medical services. Attempts have been made to address this by advanced patient scheduling techniques including estimating a likely rate of no-shows. However, these approaches do not take into account other factors that can impact wait time. Particularly, it is recognized herein that accuracy in estimating patient wait times can be improved by estimating patient-specific examination time durations for medical examinations of respective patients in the queue based ahead of the patient whose wait time is being estimated. For example, the actual wait time for a patient who has three patients ahead of him or her in the queue may be very different if those three patients are (for example) patients with normal mobility and cognition, as compared with if those three patients are (for example) patients with impaired mobility or cognition.

Furthermore, it is recognized herein that accuracy in estimating patient wait times can be improved by taking into account the workflow of the laboratory providing the medical examination. For example, in the case of an imaging examination, the wait time if there are three patients ahead in the queue may be very different if the laboratory has three operational medical imaging devices each presently staffed by an imaging technician, as compared if one of those three imaging devices is not in operation due to problems with the imaging device or having only two available imaging technicians.

The following discloses an approach for providing a wait time estimate for a given patient that is based on statistical analysis of characteristics of patients queued ahead of the given patient. A scheduled procedure can be delayed by factors such as mobility and/or cognitive impairment of a patient ahead in the queue, patient allergies (e.g. to a contrast agent to be used in an imaging procedure), a patient ahead in the queue having ancillary equipment such as an oxygen bottle or an intravenous (IV) infusion pump that may slow examination of that queue patient, a type of examination to be performed, a frequency of an examination to be performed, a likelihood of repeating an imaging session (such as, for example, due to patient movement or incorrect patient positioning), patient preparedness for examination (such as, for example, though a Medumo tracking or education system (available from Koninklijke Philips N.V., Eindhoven, the Netherlands)), issues that may result in image artefacts (e.g., metallic implants), a wellness of the patient at check-in (e.g., whether the patient has a fever), availability of additional help (such as, for example, immobile patients, patients that need to be transported or transferred, and so forth), physical monitoring of the patient such as vital signs, a predicted patient arrival time, a nature, length or instructions for a procedure or follow-up appointment, and so forth. While these factors may be statistically estimated on population level, in practice for any given patient, there are a relatively small number of patients queued ahead, and so population-level statistical estimates are of limited value.

The disclosed system includes the following components: a clinical and operational features extractor; a patient-specific examination time estimator; a patient wait time estimator; and a user interface.

The clinical and operational features extractor extracts a set of previous clinical and operational features for patients and healthcare facility (/facilities) of interest. This will allow the capture of clinical and operational information from several healthcare information systems. First, for clinical information features extraction, there could be clinical concepts and patient demographic information. Second, for operational information extraction, the information captured in this part is related to the operation information of a give clinical lab. The features are patient-specific, and the extracted features to be used in estimating wait time.

The patient-specific examination time estimator uses historical data for past patients and study information characterized by the set of features to train a machine learning (ML), statistical modeling, rule-based, or other algorithm to estimate the examination time for an input patient characterized by the set of features. The core of this module is to select features to be used in the prediction procedure time, and then use this information to compute the approximate wait time for patients. The wait time could be computed as the number of minutes to a given procedure or using a symbolic timeslot (e.g., “few minutes”, “time to order a coffee”, or “you can listen to the top three billboard music”). Features can be pre-selected before training the model or selected during model training. This examination time estimator is then applied to each patient to be examined by the laboratory to generate patient-specific examination time estimates. For example, older patients are statistically more likely to have physical mobility and/or mental cognition limitations. While this will not be the case for every patient (e.g., some 80 year-old patients have better mobility and cognition than some 50 year-old patients), but due to the statistical correlation between advanced age and compromised mobility/cognition, using patient age as a surrogate for mobility/cognition level will generally provide a more accurate wait time estimate. Likewise, a higher BMI (especially in the obese range, BMI>30) statistically correlates with reduced mobility and potentially other issues such as difficulty in loading the patient into an examination apparatus. Smokers also statistically have lower mobility, especially of the patient feature of “smoker” is combined with other patient features correlating with reduced mobility such as advanced patient age and high BMI. Patients with certain chronic illnesses such as Chronic Obstructive Pulmonary Disease (COPD) or emphysema are strongly correlated with reduced patient mobility (again, especially if co-occurring with other patient features such as advanced age or high BMI) and potentially increased examination complexity.

For a given patient, the wait time estimator uses the examination time estimates for the patients queued ahead of the given patient, and a laboratory workflow model, to estimate the wait time for the given patient. The laboratory workflow model takes into account resource factors such as the number of parallel examination paths (e.g., the number of operating MRI scanners in the case of a medical imaging laboratory, or the number of nurses on duty in the case of a blood draw lab, or so forth), on-duty staff level, remote staff availability, characteristics of the examinations (e.g. whether an imaging examination uses contrast agent, number of vials of blood to draw, and/or so forth). In one example, a Discrete Event Simulation can be used as the laboratory workflow model, although other options such as a simpler aggregation of the examination times of the patients in the queue scaled by factors for examination characteristics, on-duty staff level, or so forth is also contemplated. The wait time estimator optionally provides statistical wait times with confidence intervals or the like based on examination duration estimation error, simulation variance, and/or no-show/late-show rate of patients queued ahead of the given patient.

The user interface may be implemented in various ways, e.g. as a webpage, cellphone app, SMS messages pushed to the patient’s cellphone, a component of a laboratory scheduling program running on the receptionist workstation, and/or various combinations thereof. In some embodiments, the wait time estimator runs repeatedly, or in response to a change such as a patient appointment cancellation, a no show, or any other factor used to determine or alter the wait time. In some embodiments, the user interface outputs the wait time to the nearest 15 minutes (or nearest 10 minutes, et cetera), and/or adds buffer time (e.g., indicating a start time that is, e.g., 10 minutes before the start time estimated by the wait time estimator). A buffer time can be set by hospital managers and/or by tracking patient’s location to allow transportation time for patient to arrive. In this way, patients do not need to wait in a specified location (e.g. waiting room) to get called, which is expected to improve patient experience and satisfaction.

In some embodiments disclosed herein, the user interface is integrated with a remote check-in feature, e.g. as a cellphone app, and in such cases the patient may optionally have the ability to reschedule for another time or another lab location if the patient deems the wait too long. In another optional aspect, the patient may be able to provide information to the system via the user interface that is used to estimate wait times for those patients later in the queue. For example, the patient can indicate he/she will be 20 minutes late.

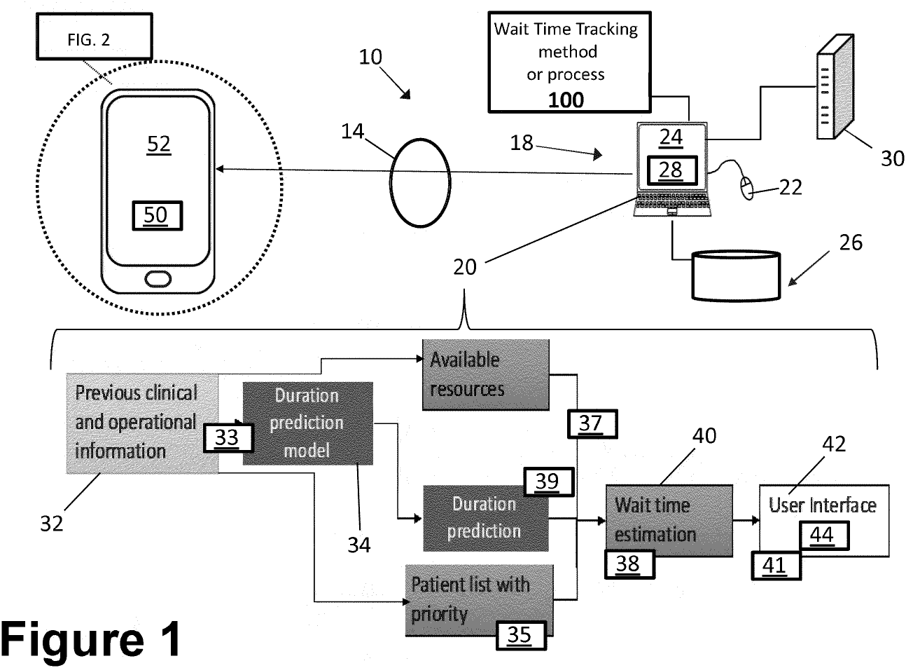

With reference to FIG. 1, an illustrative apparatus 10 is shown for tracking a wait time for a patient for a medical examination or other medical procedure. FIG. 1 also shows an electronic processing device 18, such as a workstation computer, or more generally a computer. The electronic processing device 18 typically includes a patient scheduling system, and may also include a server computer or a plurality of server computers, e.g. interconnected to form a server cluster, cloud computing resource, or so forth, to perform more complex computational tasks. The workstation 18 includes typical components, such as an electronic processor 20 (e.g., a microprocessor), at least one user input device (e.g., a mouse, a keyboard, a trackball, and/or the like) 22, and a display device 24 (e.g. an LCD display, plasma display, cathode ray tube display, and/or so forth). In some embodiments, the display device 24 can be a separate component from the workstation 18, or may include two or more display devices.

The electronic processor 20 is operatively connected with one or more non-transitory storage media 26. The non-transitory storage media 26 may, by way of non-limiting illustrative example, include one or more of a magnetic disk, RAID, or other magnetic storage medium; a solid state drive, flash drive, electronically erasable read-only memory (EEROM) or other electronic memory; an optical disk or other optical storage; various combinations thereof; or so forth; and may be for example a network storage, an internal hard drive of the workstation 18, various combinations thereof, or so forth. It is to be understood that any reference to a non-transitory medium or media 26 herein is to be broadly construed as encompassing a single medium or multiple media of the same or different types. Likewise, the electronic processor 20 may be embodied as a single electronic processor or as two or more electronic processors. The non-transitory storage media 26 stores instructions executable by the at least one electronic processor 20. The instructions include instructions to generate a visualization of a graphical user interface (GUI) 27 for display on the display device 24.

The apparatus 10 also includes, or is otherwise in operable communication with, one or more database(s) 30 storing a data related to a queue of patients with schedule medical procedures time and/or data related to operations of the medical facility where the scheduled medical procedures are to occur. The database 30 can be any suitable database, including a Radiology Information System (RIS) database, an Electronic Medical Records (EMR) database, an Electronic Health Records (EHR) database, a Cardiology Information System (CIS) database, a Health-Level 7 (HL7)-compliant database, and so forth. Alternatively, the database 30 can be implemented in the non-transitory medium or media 26.

In general, the one or more database(s) 30 include or provide the functionality of: (i) a scheduler, which maintains a schedule of appointments for the medical examination thus providing the patient queue; and (ii) a patient data repository such as an EMR or EHR that stores patient data from which the patient features are extracted. As an example of the scheduler, a radiology laboratory may have a scheduler component built into the RIS which provides for making patient appointments as well as storing the examination order for each patient. The scheduler typically stores specific appointment times for the patients of the queue of patients, and the order of patients in the queue is then determined from the patient appointment times (e.g., if patients A, B, C, D, and E have respective appointment times of 10:00 am, 10:30 am, 11:00 am, 11:30 am, and 1:00 pm, then the patients are ordered in the queue as A-B-C-D-E where A is the first patient to be seen and E is the last patient to be seen). In practice, it is often not possible to perform every patient examination at the scheduled start time, e.g. if there is only a single medical imaging device and the examination of patient B in the last example runs from 10:30 am to 11:15 am then the examinations of patients C, D, and E will be pushed back and start at times later than their scheduled appointment times. Additionally or alternatively, some or all patients may not have specified appointment times and are then otherwise ordered into the queue. For example, an emergency patient may be “fitted into” the schedule in various ways, such as by being assigned the next available examination slot. It is also contemplated in some environments to have only the queue order without specific patient appointment times, for example as may be done for a laboratory that is servicing only in-hospital patients. In this case, the queue may specify patients to be serviced in the order A-B-C-D-E but without specifying specific appointment times for the patients.

FIG. 1 also shows modules implemented by the at least one electronic processor 20 to track wait times of the schedule patients. A capture module 32 (corresponding to the previously mentioned clinical and operational features extractor) is configured to capture clinical and operational data including, for example, one or more of clinical data, data of the queued patients, and operational data of the medical facility from the database 30. The captured data can be of different data types, modules, formats, semantics, and so forth. The capture module 32 can include any suitable IT communication protocol to communicate with the database 30, such as a HL7 protocol, a Digital Imaging and Communications in Medicine (DICOM) protocol, a Fast Healthcare Interoperability Resources (FHIR) protocol, and so forth. The clinical data can be captured by the capture module 32 can be implemented by, for example, natural language processing (NLP) or a Regular Expression (RgEx) process.

The clinical data can include, for example, smoke history, presence of an echocardiogram (ECG) examination, a Left Ventricle (LV) examination, an enlarged LV examination, cardiology examinations and ultrasound imaging examinations. To enhance operability, this information can be represented using ontology procedures such as SNOMED CT and RadLex. The patient data can be found in clinical documents (e.g., clinical notes, radiology reports, medical history, and so forth) stored in the database 30, and can include age, gender, smoking history, chest pain history, diabetes history, presence of a contrast alert, vital sign data (e.g., height, weight, blood pressure, and so forth), history of chronic diseases, and so forth). The operational data, can include, for example, staff experience, equipment used, use of a contrast agent, a number of procedures performed, a location of the procedure, a type of procedure, a protocol used for a procedure, and so forth. The capture module 32 is configured to generate several outputs: prediction data 33, a prioritized patient list 35, and an available resources list 37.

The prediction data 33 captured by the capture module 32 is input to a medical procedure or examination duration prediction module 34 (corresponding to the previously mentioned patient-specific examination time estimator), and is used predict a medical procedure examination wait time 39 for on-going procedures or examinations for patients on a schedule of a medical facility (and optionally to prioritize the schedule). The duration prediction module 34 is configured to select a set of features from the data in order to generate the predicted wait time 39 for each patient in the queue for the medical examination. To do so, statistics or machine-learning (ML) processes can be used. In one example, a Pearson correlation process can be used to eliminate correlated features and reduce a number of potential features that can be used to predict the wait time. Once the set of features is selected, a supervised prediction or classification process (e.g., statistical modeling, machine learning, rule-based or deep learning, and so forth) can be used to predict the medical procedure duration. In a supervised approach, a potential feature can be an examination duration from a study profiling for each exam and the complexity of the examination as indicated by the finding codes, or manually labeled. The set of features can also include data captured from study or patient profiling. In a deep learning process, results can be predicted with multiple labels. Alternatives, multiple processes can be used to predict medical procedure duration and complexity in separate processes. In some examples, factors that may result in a delay of the patient’s appointment can be used by the duration prediction module 34 to adjust the predicted wait time 39, such as mobility and/or cognitive impairment of a patient ahead in the queue, patient allergies (e.g. to a contrast agent to be used in an imaging procedure), a patient ahead in the queue having ancillary equipment such as an oxygen bottle or an intravenous (IV) infusion pump that may slow examination of that queue patient, a type of examination to be performed, a frequency of an examination to be performed, a likelihood of repeating an imaging session (such as, for example, due to patient movement or incorrect patient positioning), patient preparedness for examination (such as, for example, though a Medumo tracking or education system), issues that may result in image artefacts (e.g., metallic implants), a wellness of the patient at check-in (e.g., whether the patient has a fever), availability of additional help (such as, for example, immobile patients, patients that need to be transported or transferred, and so forth), physical monitoring of the patient such as vital signs, a predicted patient arrival time, a nature, length or instructions for a procedure or follow-up appointment,

The predicted wait time 39 for each patient in the queue for the medical examination/procedures, along with the prioritized patient list or queue 35, and the available resources list 37, are input to a wait time estimation module 40 (corresponding to the previously mentioned wait time estimator). The wait time estimation module 40 is configured to estimate a patient wait time 41 using these inputs. In one example, the wait time estimation model 40 can generate the patient wait time 41 by using a workflow model 38, such as a discrete event simulation model, a machine-learning (ML) workflow model, a statistical workflow model, and a rule-based workflow model, among others. The workflow model 38 can be trained, for example, using historical clinical and operational features extracted from the database 30. In another example, the wait time estimation model 40 can generate the patient wait time 41 by using a simplified mathematical model such as by aggregating examination times for currently examined patients and patients with higher priority than a selected or query patient (that is, by aggregating the estimated examination time durations of patients ahead of the query patient in the queue) divided by the number parallel examination paths (which could be as low as a single examination path, e.g. in the case of medical imaging examinations to be performed by a radiology laboratory having a single working imaging device; or, which could be higher, e.g. three parallel examination paths if the radiology laboratory has three imaging devices each staffed by a respective imaging technologist and capable of handling the medical imaging examinations). In some embodiments, the patient wait time 41 can be a time range generated by calculating confidence intervals from the duration prediction module 34 and a no-show/late-show history rate of patient to be arrived with a higher priority, which can be estimated based on the captured data by the capture module 32.

A user interface module 42 is configured to generate an interface 44, which includes the patient wait time 31, for notification to the patient. The user interface module 42 is configured to send the patient wait time 41 to the patient in advance of the schedule medical procedure time (e.g., 10-30 minutes). In addition, the interface 44 can be displayed on the display device 24 of the workstation 18 (e.g., on a web page) for review by a medical professional before transmission to the patient. As shown in FIG. 1, the computer 18 is configured to send the patient wait time 41 to a mobile device 52 of the patient via a communication link 14, which typically comprises the Internet augmented by local area networks. For example, the patient can log-in into a mobile application program (“app”) 50 which is loaded on, and executable on, the mobile device 52 of the patient (e.g., an illustrative cellular telephone 52, or a tablet computer, personal data assistant or PDA, and/or so forth) to receive the patient wait time 41. The app 50 may be downloaded to the mobile device 52 from an app store accessed via a Wi-Fi, cellular, or other wireless communication network. In a suitable embodiment, the app 50 is represented on the home screen or applications screen of the mobile device 52 as an app icon (i.e. a small square, round, or other compact graphical element representing the app 50) and the user launches (i.e. initiates running of) an instance of the app 50 on the device 52 by touching the icon on a (touch-sensitive) screen of the mobile device 52.

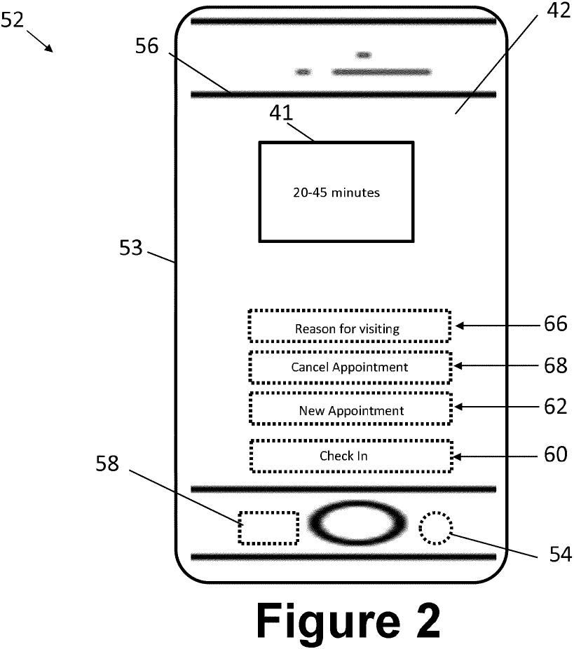

FIG. 2 shows an example of a display suitably shown by an instance of the app 50 running on a user mobile device 52 of an associated hospital staff personnel. The display is suitably shown in a touch-sensitive screen 53 of the mobile device 52. The app 50 provides the user interface 44 for display on a display device 56 of the mobile device 52. The mobile device 52 also includes at least one electronic processor 58, and optionally further includes a GPS sensor 54. Upon generating the patient wait time 41, the electronic processor 58 is programmed to receive the user interface 44 from the workstation 18, and provided on the display device 56. As shown in FIG. 2, the user interface 54 includes a plurality of fields, including (among others): (i) a field showing the patient wait time 41; a check-in field 60 allowing the patient to check in for the medical procedure, a new appointment field 62 in which the user can schedule a new appointment for the medical procedure; a new facility field 64 allowing the patient to schedule an examination at another medical facility; a reason for procedure field 66; and a cancel appointment field 68 allowing the patient to cancel the scheduled appointment.

Once the user interface 44 is displayed, the electronic processor 58 is programmed to receive a user input (e.g., a finger tap on the display device 56) from the individual of a selection of either the check-in field 60, the new appointment field 62, or the new facility field 64.

If the check-in field 60 is selected, the new appointment field 62 and the new facility field 64 are removed from the user interface 44. If the new appointment field 62 is selected, then a schedule of available times can be provided to the user on the user interface 44. If the new facility field 64 is selected, the GPS sensor 54 in the phone 52 can collect position information of the patient, and determine a location of one or more medical facilities at which the patient can schedule an appointment.

In some examples, if the patient is a new patient at the medical facility, a questionnaire or query can be provided to the patient for storage in the database 32.

The apparatus 10 is configured as described above to perform a patient wait time tracking method or process 100. The non-transitory storage medium 26 stores instructions which are readable and executable by the at least one electronic processor 20 to perform disclosed operations including performing the patient wait time tracking method or process 100. In some examples, the method 100 may be performed at least in part by cloud processing.

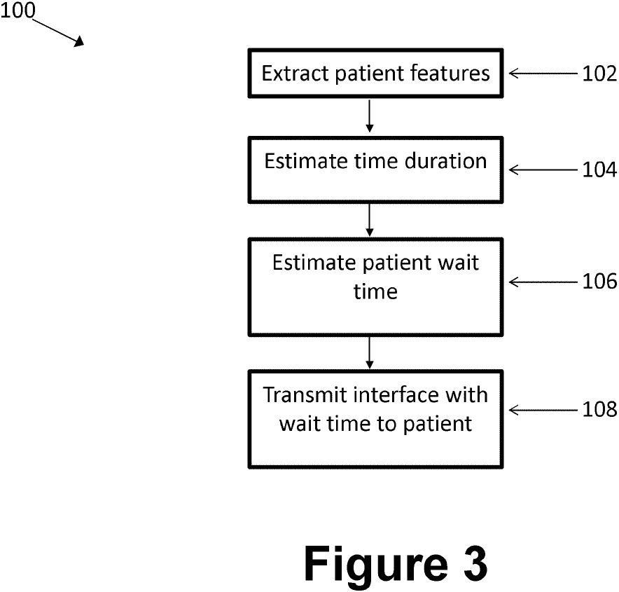

With reference to FIG. 3, an illustrative embodiment of an instance of the patient wait time tracking method 100 is diagrammatically shown as a flowchart. Although described primarily in terms of a selected or “query” patient, the operations of the method 100 are repeated for all patients in a patient queue of patients scheduled for examination. For example, if the patient queue includes patients A, B, C, and D, then the wait time 41 for patient C is based on the medical procedure time durations for patients A and B, while the wait time for D is based on the procedure time durations patients A, B, and C. At an operation 102, patient features for respective patients of a queue of patients schedule for examination are extracted. The operation 102 can be performed by the capture module 32 by retrieving the data from the database 30. In some examples, the extracted patient features can include one or more of age, gender, body mass index, smoking history, and chronic illness data, among others. In other examples, the extracted patient features can also include patient-specific examination features for the medical examinations of the respective patients of the queue. In a further example, the patient features can be received from a patient via a questionnaire or query sent to the mobile device 52 of the user. From this, the prediction data 33, the prioritized patient list 35, and the available resources list 37 are generated.

At an operation 104, patient-specific examination time durations 39 are estimated for the medical procedures or examinations of the respective patients of the queue. These time durations are estimated based on the extracted patient features of the respective patients of the queue. The operation 104 can be performed by the medical procedure duration prediction module 34. The “examination” or “procedure” time duration can mean when the procedure or examination begins (i.e., a length of the examination or procedure). In some examples, the time duration 39 can be estimated based at least in part on the one or more patient features for the patient. The patient-specific examination time durations 39 will in general be different for different patients, e.g. the estimated time duration for a patient whose patient features include “non-smoker”, “age less than <threshold>”, and negative values for chronic conditions such as COPD will be shorter than the estimated time duration for a patient whose patient features include “smoker”, “age greater than <threshold>”, and positive values for chronic conditions such as COPD.

At an operation 106, a wait time 41 is estimated for a query patient of the patients of the queue based on the estimated examination time durations of patients ahead of the query patient in the queue (e.g., a wait time for query patient C will be different than for patient D). The operation 106 can be performed by the wait time estimation module 40. To do so, the workflow model 38 is applied for the medical examinations or procedure of the respective patients of the queue. The workflow model 38 represents a number of concurrent examinations or procedures (e.g., a number of operating imaging devices for imaging examinations, a number of medical personnel available, and so forth). The workflow model 38 can be for example, a ML workflow model, a statistical workflow model, a rule-based workflow model, a Discrete Event Simulation model, among others. In some examples, a confidence interval can be calculated for the patient wait time 41.

At an operation 108, the patient wait time 41 is transferred to a user interface 44 for presentation. The operation 108 can be performed by the user interface module 42. In one example, the transmitting of the user interface 44 (including the patient wait time 41) can include transmitting the user interface to a smart device 52 of the query patient via an email, SMS message, the app 50, and so forth. In another example, the transmitting of the user interface 44 (including the patient wait time 41) can include transmitting the user interface to a web browser running a webpage configured to display the estimated wait time 41. In some embodiments, the patient wait time 41 can be displayed on the user interface 44 as a running timer, or alternatively can be rounded to a nearest time interval (e.g., 15 minute time intervals), and can be updated to include a buffer time period (e.g., 15 minutes before the wait time 41 countdown begins).

The operations 102-108 can be iteratively repeated for the query patient to iteratively update the estimated wait time 41 for the query patient.

The disclosure has been described with reference to the preferred embodiments. Modifications and alterations may occur to others upon reading and understanding the preceding detailed description. It is intended that the disclosure be construed as including all such modifications and alterations insofar as they come within the scope of the appended claims or the equivalents thereof.

1. A non-transitory computer readable medium storing instructions executable by at least one electronic processor to perform a patient wait time tracking method, the method comprising:
extracting clinical and operational features for respective patients of a queue of patients scheduled for examination;
estimating patient-specific examination time durations for medical examinations or procedures of the respective patients of the queue based on the extracted clinical and operational features of the respective patients of the queue;
estimating a wait time for a query patient of the patients of the queue based on the estimated examination time durations of patients ahead of the query patient in the queue; and
transmitting the estimated wait time to a user interface for presentation.

2. The non-transitory computer readable medium of claim 1, wherein estimating a wait time for the query patient includes:
applying a workflow model for the medical examinations of the respective patients of the queue.

3. The non-transitory computer readable medium of claim 2, wherein the workflow model represents one or more of:
a number of concurrent examinations, and/or a number of available medical resources.

4. The non-transitory computer readable medium of claim 1, wherein the time duration prediction comprises one of a machine-learning (ML), a statistical workflow model, a rule-based model and a deep learning model.

5. The non-transitory computer readable medium of claim 1, wherein estimating patient-specific examination time durations includes estimating the patient-specific examination wait times based on one or more of: mobility and/or cognitive impairment of a patient ahead in the queue, patient allergies, a patient ahead in a queue having ancillary equipment that may slow examination, a type of examination to be performed, a frequency of an examination to be performed, a likelihood of repeating an imaging session, patient preparedness for examination, issues that may result in image artefacts, a wellness of the patient at check-in, availability of additional help, physical monitoring of the patient, a predicted patient arrival time, and a nature, length or instructions for a procedure or follow-up appointment.

6. The non-transitory computer readable medium of claim 2, wherein the workflow model is a Discrete Event Simulation model.

7. The non-transitory computer readable medium of claim 1, wherein estimating the wait time further includes:
calculating a confidence interval for the estimated wait time.

8. The non-transitory computer readable medium of claim 1, wherein the extracted clinical and operational features include one or more of age, gender, body mass index, smoking history, and chronic illness data from a database.

9. The non-transitory computer readable medium of claim 1, wherein the extracted clinical and operational features include patient-specific examination features for the medical examinations of the respective patients of the queue.

10. The non-transitory computer readable medium of claim 1, wherein the transmitting includes at least one of:
transmitting the estimated wait time to a cellphone of the query patient via an email, automatic phone call, or SMS message;
transmitting the estimated wait time to a web browser running a webpage configured to display the estimated wait time.

11. The non-transitory computer readable medium of claim 1, wherein the transmitting includes at least one of:
displaying the estimated wait time as a running timer; and
rounding the wait time to a nearest time interval.

12. The non-transitory computer readable medium of claim 1, wherein the transmitting includes:
iteratively repeating the extracting, the estimating of patient-specific examination time durations, and the estimating of the wait time for the query patient to iteratively update the estimated wait time for the query patient.

13. The non-transitory computer readable medium of claim 1, wherein the method further includes:
updating a display of the estimated wait time on the user interface to include a buffer time period.

14. The non-transitory computer readable medium of 1, wherein the transmitting further includes:
receiving a check-in for the query patient via the user interface.

15. The non-transitory computer readable medium of claim 1, wherein the extracting includes:
receiving one or more clinical and operational features for the query patient via the user interface, wherein the patient-specific examination time duration for the medical examination of the query patient is estimated based at least in part on the one or more patient features for the query patient received via the user interface.

16. An apparatus, comprising:
a database storing patient data related to patients of a queue of patients scheduled for examination;
a display device upon which a user interface is implemented; and
at least one electronic processor programmed to:
extract patient features for the respective patients of the queue of patients from the database;
estimate patient-specific examination time durations for medical examinations of the respective patients of the queue based on the extracted patient features of the respective patients of the queue;
estimate a wait time for a query patient of the patients of the queue based on the estimated examination time durations of patients ahead of the query patient in the queue; and
transmit the estimated wait time to the user interface for presentation.

17. The apparatus of claim 16, wherein the at least one electronic processor is further programmed to:
apply a workflow model for the medical examinations of the respective patients of the queue to estimate the wait time.

18. The apparatus of claim 16, wherein the extracted patient features include patient-specific examination features for the medical examinations of the respective patients of the queue.

19. The apparatus of claim 16, wherein the transmitting includes at least one of:
transmitting the estimated wait time to a cellphone of the query patient via an email or SMS message;
transmitting the estimated wait time to a web browser running a webpage configured to display the estimated wait time.

20. A patient wait time tracking method, comprising:
extracting clinical and operational features for respective patients of a queue of patients scheduled for examination;
estimating patient-specific examination time durations for medical examinations of the respective patients of the queue based on the extracted clinical and operational features of the respective patients of the queue;
applying a workflow model for the medical examinations of the respective patients of the queue;
estimating a wait time for a query patient of the patients of the queue based on the estimated examination time durations of patients ahead of the query patient in the queue and the workflow model; and
transmitting the estimated wait time to a user interface for presentation.
